(19) United States
(12) Patent Application Publication
(10) Pub. No.: US 2021/0002341 A1
(43) Pub. Date: Jan. 7, 2021

(54) METHOD FOR REMOVING N-TERMINAL TRUNCATED AND ABNORMAL VARIANTS IN RHNGF

(71) Applicant: Xintrum Pharmaceuticals, Ltd., Nanjing (CN)

(72) Inventors: Wenchao Liu, Nanjing (CN); Hongliang Sun, Nanjing (CN); Yi Zhang, Nanjing (CN); Yuesheng Wang, Nanjing (CN)

(21) Appl. No.: 17/030,306

(22) Filed: Sep. 23, 2020

Related U.S. Application Data
(63) Continuation of application No. PCT/CN2018/114563, filed on Nov. 8, 2018.

(30) Foreign Application Priority Data
Mar. 26, 2018 (CN) 201810253680.6

(51) Int. Cl. C07K 14/48; C07K 1/36

(52) U.S. Cl. CPC C07K 14/48; C07K 1/36

(57) ABSTRACT

A method for removing an N-terminal truncated variant and an abnormal variant in recombinant human nerve growth factor (rhNGF) is provided. An rhNGF raw material loaded on a cation-exchange material is washed with a washing liquid to obtain a washed raw material from which an N-terminal truncated variant and an abnormal variant have been removed, where the washing liquid has higher electrical conductivity than the rhNGF raw material. Cation-exchange chromatography (CEC) elution is then performed on the washed raw material with an elution buffer having higher electrical conductivity than the washing liquid. A purified rhNGF product is obtained from the eluate.

### TECHNICAL FIELD

The present invention relates to a method for removing N-terminal truncated variants and abnormal variants in recombinant human nerve growth factor (rhNGF) and more particularly to a method for removing such N-terminal truncated and abnormal variants by increasing electrical conductivity in stages.

### DESCRIPTION OF RELATED ART

rhNGF expressed by a Chinese hamster ovary (CHO) cell, which is an eukaryotic expression system, tends to contain variants. The term “variant” as used herein refers to any of a series of proteins that are formed by post-translational modification during intracellular secretion, or by the chemical reaction of an amino acid residue side chain after secretion, or by the degradation of a peptide chain.

rhNGF is synthesized in vivo in the form of a precursor. Incomplete processing with furin or prohormone convertase leads to the formation of complete precursors and partial precursors, which are collectively referred to herein as precursor variants. Apart from precursors, variants such as N-terminal truncated variants, oxidized variants, deamidated variants, isomeric variants, C-terminal truncated variants, and abnormal variants may also be produced due to the properties of eukaryotes.

An “N-terminal truncated” variant refers to a sequence molecule whose N terminal lacks certain amino acids as a result of post-translational processing. Herein, an “N-terminal truncated” variant refers particularly to a 6˜117 sequence molecule.

“Abnormal variants” is a collective term for variants with structural abnormality (with the hydrophobic core exposed), disulfide bond abnormality, or abnormal oxidation (with the hydrophobic core sites oxidized) as a result of post-translational processing. Generally, abnormal variants appear later than the main peak of 1˜117 in a reversed-phase high-performance liquid chromatography (RP-HPLC) analysis but earlier than the main peak of 1˜117 in a weak cation-exchange high-performance liquid chromatography (WCX-HPLC) analysis.

The chromatography methods in the prior art, though capable of removing many process-related impurities in rhNGF (e.g., host cell proteins and nucleic acids), have difficulties in removing rhNGF variants that emerge as product-related impurities, in which “N-terminal truncated” variants and “abnormal” variants are the major types of rhNGF variants. As these variants are generally produced along with mature rhNGF and have similar physical and chemical properties to the rhNGF product, it is difficult to purify rhNGF on a large scale.

Currently, reports on the purification of rhNGF include the following:

Chinese Published Patent Application No. 102702341A uses a two-step method that involves cation exchange and a molecular sieve (Superdex 75) to prepare an rhNGF whose purity is higher than 98%; the cation exchange step, however, is used only to remove such process-related impurities as host cell proteins. Chinese Published Patent Application No. 106478801A uses a two-step method that involves cation exchange and hydrophobic interaction chromatography (HIC) (preferably involving the use of the phenyl group) to prepare an rhNGF whose purity is higher than 99%, and the cation exchange step is used to capture the intended product and remove such process-related impurities as host cell proteins, too.

Chinese Patent No. 1268639C uses high-performance cation exchange and linear gradient elution to separate oxidized, isomeric, or deamidated rhNGF variants with relatively good results.

None of the foregoing methods involves, let alone can remove, N-terminal truncated or abnormal variants; moreover, linear gradient elution is used in the chromatography step. Linear gradient elution generally necessitates a two-pump chromatography system and therefore has rather strict requirements for the equipment, which is nevertheless disadvantageous to large-scale industrial production.

### SUMMARY OF THE INVENTION

One objective of the present invention is to remove an N-terminal truncated variant and an abnormal variant in rhNGF.

N-terminal truncated variants and abnormal variants are the most detrimental impurities to the quality of rhNGF and therefore must be removed.

The inventors of the present invention analyzed the physical and chemical properties of rhNGF and its variants and has found that N-terminal truncated variants and abnormal variants peak before the main peak in a WCX-HPLC analysis, meaning those variants have relatively low isoelectric points. The cation-exchange chromatography (CEC)-based purification process of the present invention, therefore, removes N-terminal truncated variants and abnormal variants by increasing electrical conductivity in stages, which proved to be effective.

The operation method is detailed as follows:

A method for removing an N-terminal truncated variant and an abnormal variant in rhNGF is characterized by including the steps of:

1) washing with a washing liquid the rhNGF raw material loaded on a cation-exchange material, and thereby obtaining a washed raw material from which an N-terminal truncated variant and an abnormal variant have been removed, wherein the washing liquid is a washing buffer having higher electrical conductivity than the rhNGF raw material; and

2) performing CEC elution on the washed raw material in step 1) with an elution buffer having higher electrical conductivity than the washing liquid in step 1), and collecting the eluate in order to obtain a pure rhNGF product from the eluate.

The electrical conductivity of the washing liquid in step 1) is 20˜30 mS/cm.

The washing liquid in step 1) is an NaCl-containing buffer with an NaCl content of 200˜300 mM and a pH value within the same pH range as the rhNGF raw material, generally 5.5˜6.5.

The washing volume is 7˜10 column volume (CV), preferably 8 CV.

The process of step 1) includes loading the cation-exchange material with the rhNGF raw material, washing with the washing liquid, and discarding the outflowing liquid.

The rhNGF raw material in step 1) is a preliminarily purified product obtained by subjecting a CHO cell culture to column chromatography once or for multiple times. The CHO cell culture is the rhNGF expressed by a cell culture of CHO-cell-recombination host cells.

The preliminarily purified product, although having been subjected to column chromatography purification by a prior art method at least once, still contains rhNGF variants (e.g., N-terminal truncated variants, precursors, and abnormal variants) and a large amount of other contaminants that are difficult to remove with the conventional means. The present invention has no limitation on the column chromatography method employed. All the column chromatography methods well known to a person skilled in the art (e.g., HIC, anion-exchange chromatography, CEC, and mixed-mode ion-exchange chromatography) can be used.

The elution buffer used in step 2) is an NaCl-containing buffer, and the elution buffer should satisfy the following conditions at the same time:

A. having higher electrical conductivity than the washing liquid in step 1); and

B. having an NaCl content of 350˜600 mM.

The electrical conductivity of the elution buffer is 35˜60 mS/cm.

The buffer salt used in the washing liquid and the elution buffer is selected from sodium acetate, phosphates, MES, and MOPSO.

The aforesaid electrical conductivity can be adjusted by adding a salt, and the salt is selected from sodium chloride, potassium chloride, sodium sulfate, and sodium acetate.

The chromatography medium has the sulfopropyl group as the cation-exchange ligand.

The term “washing” refers to allowing a washing buffer to flow through a cation-exchange material and discarding the outflowing liquid (which carries some impurities away).

The term “elution” refers to allowing an elution buffer to flow through a cation-exchange material and collecting the outflowing liquid (which contains the purified target product).

The inventors of the present invention studied the materials used in chromatography. The cation-exchange materials with which the inventors have experimented for the present invention include highly cross-linked agarose-based solid phases (e.g., SP HP from GE) and styrene-divinylbenzene-based solid phases (e.g., the POROS 50HS column from Applied Biosystems). Solid-phase cation-exchange materials with relatively large particle sizes such as Capto S from GE are not very effective in removing rhNGF variants. It was found through experimentation that the cation-exchange ligand of the chromatography medium is preferably the sulfopropyl group.

In one embodiment of the present invention, the cation-exchange purification method generally includes the steps, to be sequentially performed, of: (1) equilibrating a cation-exchange material; (2) loading the cation-exchange material with a composition; (3) performing overhead washing with an equilibration buffer; (4) performing intermediate washing with a washing buffer; and (5) eluting with an elution buffer to obtain the desired purified rhNGF product.

Generally, the equilibration buffer is allowed to flow through the cation-exchange material before the cation-exchange material is loaded with a composition that contains rhNGF and one or more molecular variants of rhNGF. In one preferred embodiment of the present invention, the equilibration buffer has a pH value of about 5.5 to about 6.5, such as about 6.2. An illustrative equilibration buffer contains 20 mM MES and 110 mM NaCl and has a pH value of 6.2.

Once equilibrium is achieved, the cation-exchange material is loaded with the composition, which contains rhNGF and one or more molecular variants of rhNGF. The composition has a pH value ranging from 5.5 to 6.5, such as 5.8 or 6.2, and electrical conductivity ranging from 10 to 14 mS/cm, such as 13 mS/cm. In one embodiment, the cation-exchange material is loaded with a composition obtained from HIC elution, and the loading density is about 1˜5 g/L resin in order for rhNGF and its variants to bind to the cation-exchange filler while most of the host cell proteins (HCP) flow through the filler.

After loading, overhead washing is carried with the equilibration buffer. The overhead washing conditions are identical to the conditions of the equilibration step. Generally, the overhead washing volume is 2˜3 times the column volume.

When overhead washing is completed, the cation-exchange material is washed with the washing buffer. During the washing process, the washing buffer flows through the cation-exchange material. The composition of the washing buffer is generally so chosen as to elute as large an amount of molecular variants (e.g., N-terminal truncated variants and abnormal variants) from the resin as possible, but not to elute the desired rhNGF. The pH value of the washing buffer is controlled between 5.5 and 6.5, such as at about 5.8 or 6.2, and the electrical conductivity of the washing buffer is controlled between 20 and 30 mS/cm, such as at about 29 mS/cm. Buffer salts that provide buffering in the aforesaid pH range include but are not limited to MES, MOPSO, sodium acetate, and phosphates. It is preferable that the washing buffer contains 20 mM MES and 290 mM NaCl and has a pH value of 5.8, or that the washing buffer contains 20 mM PB and 220 mM NaCl and has a pH value of 6.2.

After the washing step, the desired rhNGF is eluted from the cation-exchange material. The elution of rhNGF can be achieved by increasing electrical conductivity or ionic strength. The electrical conductivity of the elution buffer must be higher than about 35 mS/cm, and an increase in electrical conductivity can be attained by providing the elution buffer with a relatively high salt concentration. Salts that can be used for this purpose include but are not limited to sodium chloride, potassium chloride, and sodium acetate. In one embodiment, the elution buffer contains about 350 to about 6000 mM NaCl. In most cases, the elution buffer has generally the same pH value as the washing buffer. One preferred elution buffer contains 20 mM MES and 0.4 M NaCl and has a pH value of 6.2. Another preferred elution buffer contains 20 mM PB and 0.5 M NaCl and has a pH value of 6.2.

While the cation-exchange purification method disclosed herein may include other steps, it is preferable that the method is composed only of the following steps: equilibration; loading of the composition, which contains rhNGF and its molecular variants; the washing step for eluting the molecular variants; and the elution step for eluting the rhNGF.

If necessary, the rhNGF preparation obtained by the CEC method disclosed herein may be further purified. Illustrative further purification steps have been discussed above.

The present invention has the following advantages:

The stepwise washing+elution approach is different from the linear gradient elution in the prior art; and

Molecular variants are removed by increasing electrical conductivity in stages (i.e., the washing buffer used in the washing stage has higher electrical conductivity than the crude product to be purified, and the elution buffer used in the elution stage has even higher electrical conductivity than the washing buffer).

Experiments have proved that the method of the present invention is highly effective in removing N-terminal truncated (6˜117) molecular variants and abnormal molecular variants (see the embodiment described further below).

### BRIEF DESCRIPTION OF THE SEVERAL VIEWS OF THE DRAWINGS

FIG. 1 and FIG. 2 provide a comparison between the variant removal abilities of two fillers, namely Capto S and SP HP. The comparison between the variant (N-terminal truncated variant and abnormal variant) removal abilities of the two ion-exchange materials reveals that the variant removal ability of SP HP is superior to that of Capto S.

FIG. 3 shows a process for purifying rhNGF by CEC. The plot provides a CEC-based purification process, which is generally divided into equilibration, loading, washing, and elution.

FIG. 4 shows a comparison between the RP-HPLC analysis results of a washed sample and an eluted sample in the CEC-based purification process. The plot provides the RP-HPLC analysis results of samples taken from the CEC process. The analysis results show that N-terminal truncated variants and abnormal variants were removed by the washing process.

FIG. 5 shows a summary of variant removal rates and sample recovery rates. The plot provides the statistical analysis results of multiple batches of CEC-based purification. The analysis results show high variant removal rates and high product recovery rates, indicating that the present invention has good process performance

### DETAILED DESCRIPTION OF THE INVENTION

The following embodiment serves only to demonstrate the method and apparatus of the present invention and is not intended to be restrictive of the scope of the present invention.

The technical terms used herein are defined as follows: “1-118”, “1-117”, and “6-117” refer to different sequence molecules of rhNGF. “1-118” refers to a sequence molecule including the 1^{st} to the 118^{th} amino acids, “1-117” refers to a sequence molecule including only the 1^{st} to the 117^{th} amino acids, and “6-117” refers to a sequence molecule including only the 6^{th} to the 117^{th} amino acids.

“Contaminant” refers to any process-related impurity that is different from the desired rhNGF. A contaminant may be, but is not limited to: a substance in a host cell, such as a protein or nucleic acid of a CHO cell; endotoxin; a viral contaminant; and an ingredient of a cell culture medium.

“Cation-exchange material” refers to a solid phase that is negatively charged and has free cations to be exchanged with the cations in an aqueous solution that flows through the solid phase. Commercially available cation-exchange materials include agarose with an immobilized sulfopropyl group (SP) or sulfonyl group (S), cross-linked styrene-divinylbenzene-based solid-phase particles that are coated with a sulfopropylated and polyhydroxylated polymer, and so on.

“Load” refers to a composition loaded on a cation-exchange material.

“Equilibration buffer” refers to a buffer that is used to equilibrate a cation-exchange material before the cation-exchange material is loaded with a composition.

A “regeneration buffer” can be used to regenerate a cation-exchange filler so that the filler can be used again. The electrical conductivity and pH value of a regeneration buffer enable the buffer to remove virtually all the contaminants and rhNGF on a cation-exchange filler.

“Electrical conductivity” refers to the ability of an aqueous solution to conduct electric current between two electrodes. The electrical conductivity of a solution can be changed by varying the ion concentration of the solution.

“Overhead washing” refers to the process of washing a cation-exchange column with an equilibration buffer after the column is loaded with a composition, the objective being to wash the composition out of the column.

MES is 2-(N-morpholino)ethanesulfonic acid. MOPSO is 3-(N-morpholino)-2-hydroxypropanesulfonic acid. RP-HPLC is reversed-phase high-performance liquid chromatography. WCX-HPLC is weak cation-exchange high-performance liquid chromatography. PB refers to a phosphate buffer. TFA is trifluoroacetic acid.

Embodiment 1: CEC of rhNGF
1.1 This Embodiment Provides a CEC-Based rhNGF Purification Process.

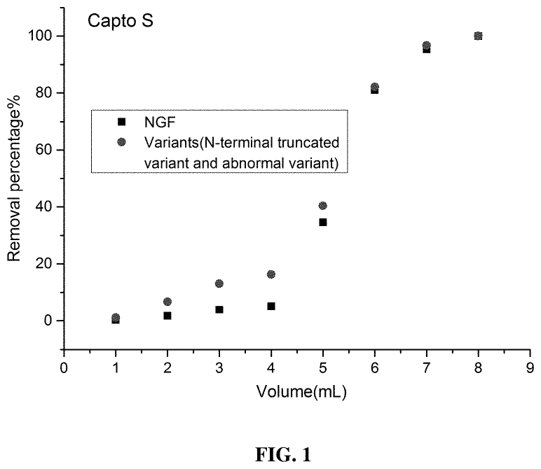

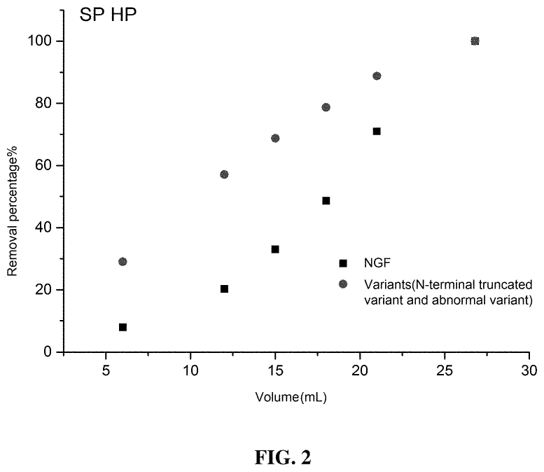

This embodiment summarizes some developmental studies on improved cation exchange steps for rhNGF. In these studies, two cation-exchange materials, namely Capto S and SP Sepharose High Performance, were evaluated in terms of their abilities to remove molecular variants (N-terminal truncated variants and abnormal variants) of rhNGF. SP Sepharose High Performance was found to have outstanding process performance in removing molecular variants of rhNGF (see FIG. 1 and FIG. 2) and was therefore used as an improved rhNGF-purifying cation-exchange resin.

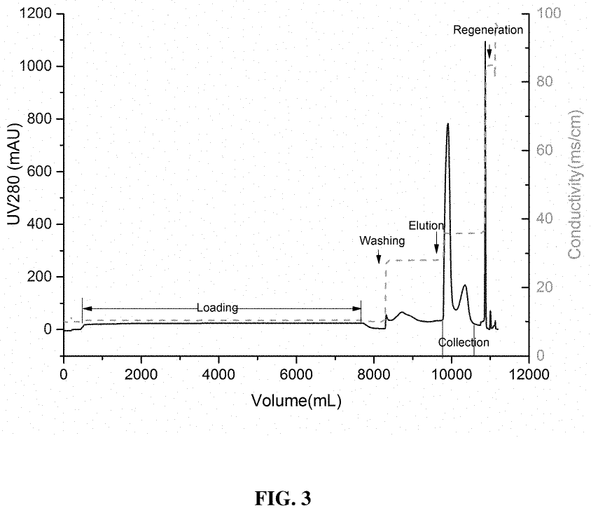

A chromatography column was operated in the binding-eluting mode at ambient temperature. The chromatography column used SP Sepharose High Performance (which is a resin composed of a highly cross-linked agarose matrix coupled with a negatively charged functional group) as the cation-exchange resin and was filled with the cation-exchange resin to a bed height of 9˜11 cm. Before loading with an HIC eluted product, the storage liquid in the cation-exchange column was washed away with an equilibration buffer, which also equilibrated the column The equilibrated chromatography column was then loaded with the HIC eluted product in order for the product to bind to the resin. After loading, overhead washing was carried out with the equilibration buffer to wash off the unbound load. Once the overhead washing was completed, the column was washed with a washing buffer to remove molecular variants. Then, elution was performed with an elution buffer having higher electrical conductivity than the washing buffer, with the volume of the elution buffer being 5 CV at most, and the eluted product was collected. After elution, the column was cleaned with a regeneration buffer (1 M NaCl) and a cleaning liquid (0.5 N NaOH) and was subsequently stored in the storage liquid until the next use (see FIG. 3).

The following table describes the process conditions of the CEC process of rhNGF according to the present invention of the present invention.

TABLE 1
the CEC process of rhNGF

| | | | Flow |
|---|---|---|---|
| | | Process | velocity |
| Stage | Buffer/solution | parameter | (cm/hr) |
| Column bed | N/A | 10 cm | N/A |
| height | | | |
| Equilibration | 20 mM MES/110 mM NaCl, pH | 4 CV | 100 |
| | 6.2 | | |
| Loading | Eluted product obtained by | 2~5 g | 100 |
| | HIC, pH 6.2, with electrical | rhNGF/L | |
| | conductivity lower than 13 | resin | |
| | mS/cm | | |
| Overhead | 20 mM MES/110 mM NaCl, pH | 2 CV | 100 |
| washing | 6.2 | | |
| Washing | 20 mM MES/220 mM NaCl, pH | 8 CV | 100 |
| | 6.2 | | |
| Elution | 20 mM MES/400 mM NaCl, pH | 5 CV | 100 |
| | 6.2 | | |
| | Start of product collection | UV280 slope | N/A |
| | | greater than 30 | |
| | End of product collection | UV280 lower | N/A |
| | | than 40 mAU | |
| Regeneration | 1M NaCl, electrical | 2 CV | 100 |
| | conductivity 84 mS/cm | | |
| Cleaning | 0.5N NaOH | 3 CV | 50 |
| Storage | 0.2M NaAc/20% ethanol | 2 CV | 50 |

### 1.2 Analysis of Purified Product

The rhNGF recovery rate and the molecular variant removal rate were analyzed by the RP-HPLC method. More specifically:

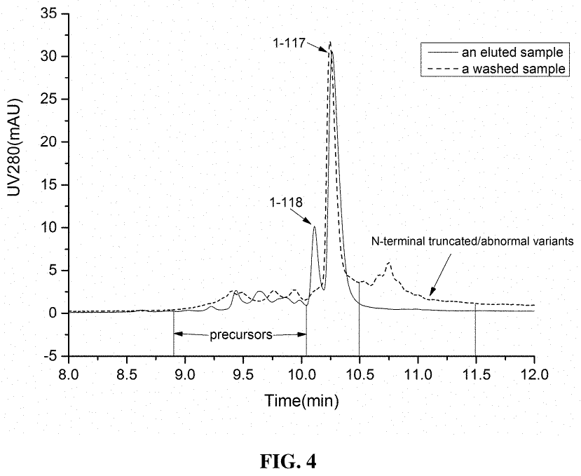

The analysis was performed with the Thermo UltiMate 3000 Dual HPLC system. The chromatography column used was Agilent C3RRHD (2.1×100 mm). Mobile phase A was an aqueous solution containing 0.1% TFA, and mobile phase B was an acetonitrile solution containing 0.1% TFA. The gradient based on the proportion of phase A was 95% at 0 min, 95% at 2 min, 73% at 4 min, 63% at 16 min, 5% at 18 min, 5% at 20 min, 95% at 22 min, and 95% at 24 min Flow velocity was 0.5 mL/min, and the detection wavelength was 280/214 nm. The proportions were calculated by the area normalization method. As an rhNGF molecule is composed of two subunits (peptide chains) that are bonded together in a non-covalent manner, and the two subunits will be dissociated in a reversed-phase analysis due to the existence of an organic solvent, the peaks on the chromatogram corresponded to the types of the subunits respectively. RP-HPLC analysis was conducted on a washed sample and an eluted sample taken from the purification process. The analysis results are plotted in FIG. 4, which shows the difference between the washed sample and the eluted sample in terms of N-terminal truncated variants and abnormal variants. The N-terminal truncated variant and abnormal variant content of the product was greatly reduced by the purification method of the present invention.

### 1.3 Statistical Data Analysis

The variant removal rate and the product recovery rate were calculated as follows, based on the RP-HPLC analysis results of the to-be-loaded composition and the eluted product:

Variant removal rate=(1−the proportion of variants in the eluted product/the proportion of variants in the to-be-loaded composition)×100%; and

Product recovery rate=(main peak area of the eluted product per unit sample input amountxeluting volume)/(main peak area of the to-be-loaded composition per unit sample input amountxloaded sample volume)×100%. The data of multiple batches of CEC-based purification was analyzed.

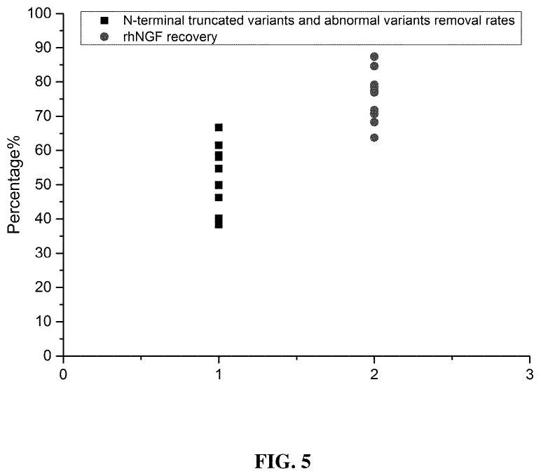

The analysis results show a variant removal rate of 52%±9% and a product recovery rate of 76%±7%, as shown in FIG. 5.

Conclusion: The method of the present invention has good process performance.

What is claimed is:

1. A method for removing an N-terminal truncated variant and an abnormal variant in recombinant human nerve growth factor (rhNGF), comprising:
1) washing with a washing liquid an rhNGF raw material loaded on a cation-exchange material, thereby obtaining a washed raw material from which an N-terminal truncated variant and an abnormal variant have been removed, wherein said washing liquid is a washing buffer having higher electrical conductivity than the rhNGF raw material; and
2) performing cation-exchange chromatography (CEC) elution on the washed raw material of step 1) with an elution buffer having higher electrical conductivity than the washing liquid in step 1), and collecting an eluate from which a purified rhNGF product is obtained.

2. The method of claim 1, wherein the electrical conductivity of said washing liquid in step 1) is 20˜30 mS/cm.

3. The method of claim 1, wherein said washing liquid in step 1) is an NaCl-containing buffer with an NaCl content of 200˜300 mM.

4. The method of claim 1, wherein step 1) comprises: loading the cation-exchange material with said rhNGF raw material, washing with the washing liquid, and discarding a resulting outflowing liquid.

5. The method of claim 1, wherein said rhNGF raw material in step 1) is a preliminarily purified product obtained by subjecting a Chinese hamster ovary (CHO) cell culture to column chromatography once or for multiple times.

6. The method of claim 1, wherein the elution buffer used in step 2) is an NaCl-containing buffer, and the elution buffer simultaneously satisfies the following conditions:
A. having higher electrical conductivity than the washing liquid in step 1); and
B. having an NaCl content of 350˜600 mM.

7. The method of claim 6, wherein the electrical conductivity of the elution buffer is 35˜60 mS/cm.

8. The method of claim 1 or 3, wherein the washing liquid and the elution buffer use a buffer salt selected from the group consisting of sodium acetate, phosphates, 2-(N-morpholino)ethanesulfonic acid (MES), and 3-(N-morpholino)-2-hydroxypropanesulfonic acid (MOPSO).

9. The method of any of claims 1 to 7, comprising adjusting said electrical conductivity by adding a salt selected from the group consisting of sodium chloride, potassium chloride, sodium sulfate, and sodium acetate.

10. The method of claim 1, wherein a chromatography medium with a cation-exchange ligand is used, and the cation-exchange ligand is the sulfopropyl group.
